(19) United States
(12) Patent Application Publication
(10) Pub. No.: US 2017/0088535 A1
(43) Pub. Date: Mar. 30, 2017

(54) CATALYSTS AND METHODS FOR MAKING CYCLIC CARBONATES

(71) Applicant: EAST CHINA UNIVERSITY OF SCIENCE AND TECHNOLOGY, Shanghai (CN)

(72) Inventors: Zhenshan HOU, Shanghai (CN); Baoning SONG, Shanghai (CN)

(73) Assignee: EAST CHINA UNIVERSITY OF SCIENCE AND TECHNOLOGY, Shanghai (CN)

(21) Appl. No.: 15/310,760

(22) PCT Filed: May 14, 2014
(86) PCT No.: PCT/CN2014/077472

§ 371 Date: Nov. 12, 2016

(51) Int. Cl. C07D 317/36; B01J 38/48; B01J 37/04; B01J 31/08; B01J 31/26; B01J 37/00; B01J 38/00; B01J 31/40

(52) U.S. Cl. CPC C07D 317/36; B01J 38/00; B01J 38/48; B01J 31/4084; B01J 31/4007; B01J 31/08; B01J 31/26; B01J 37/0036; B01J 37/04

(57) ABSTRACT

Catalysts and methods for making cyclic carbonates are disclosed. The catalyst may include at least one polymer quaternary ammonium salt, at least one metal halide and silica gel. The method of making the cyclic carbonates may include forming a mixture that includes the catalyst and an epoxide, and contacting the mixture with carbon dioxide in a reactor under conditions to form the propylene carbonate.

Unless otherwise indicated herein, the materials described in this section are not prior art to the claims in this application and are not admitted to be prior art by inclusion in this section.

Cyclic carbonates can be used in many fields. They are applicable to use as lithium battery electrolytes, polar aprotic solvents, intermediates of fine chemicals, and other fields requiring cyclic carbonates known to those skilled in the art. Ring-opening reactions of cyclic carbonates can be used to synthesize polymers such as polycarbonates, and polyurethanes. Methods for synthesizing cyclic carbonates have previously used highly toxic phosgene and can result in products containing highly corrosive hydrogen chloride. However, due to environmental issues, replacing phosgene can lead to major environmental benefits. As carbon dioxide is a major greenhouse gas, the use of carbon dioxide as a raw material in the synthesis of cyclic carbonates can solve environmental problems such as global warming Carbon dioxide can also be an economical source of raw material due to the low price of carbon dioxide.

### SUMMARY OF THE INVENTION

The foregoing summary is illustrative only and is not intended to be in any way limiting. In addition to the illustrative aspects, embodiments, and features described above, further aspects, embodiments, and features will become apparent by reference to the drawings and the following detailed description.

In a first aspect, a catalyst is described. The catalyst can comprise at least one polymer quaternary ammonium salt, at least one metal halide, and silica gel.

In a second aspect, a method of making a catalyst is described. The method can comprise incubating a first mixture comprising at least one polymer quaternary ammonium salt, at least one metal halide and a solvent, adding silica gel to the first mixture to form a second mixture, incubating the second mixture, and removing the solvent from the second mixture to obtain the catalyst.

In a third aspect, a method of making a cyclic carbonate is described. The method can comprise providing a catalyst, forming a mixture comprising the catalyst and an epoxide, and contacting the mixture with carbon dioxide in a reactor under conditions to form the cyclic carbonate. The catalyst can comprise at least one polymer quaternary ammonium salt, at least one metal halide, and silica gel.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope; the disclosure will be described with additional specificity and detail through use of the accompanying drawings.

FIG. 1 shows an exemplary route for the synthesis of propylene carbonate.

FIG. 2 shows a graphical representation of the recycling performance of a catalyst in accordance with the disclosed embodiments from a first run to a tenth run. The x-axis represents the number of times the catalyst is used and the y-axis represents conversion of propylene oxide to propylene carbonate (black solid bars) and selectivity of the propylene carbonate (diagonal striped bars).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

### Cyclic Carbonates

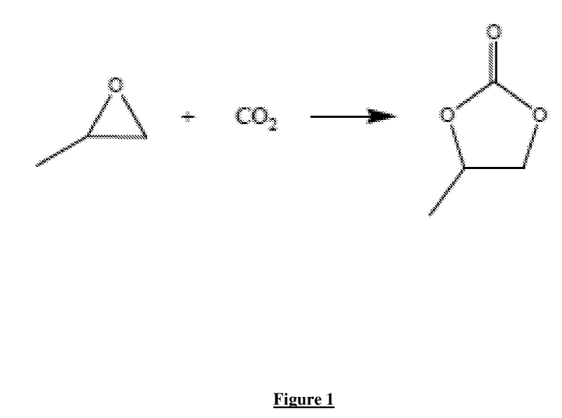

“Cyclic carbonates” as described herein, refers to any five membered alkylene carbonates. Attention is drawn to a cyclic carbonate, as shown in FIG. 1. In particular, cyclic carbonates are known for their variety of applications and have been a topic for many research projects. They can be used as excellent dipolar aprotic solvents, electrolytes for batteries, precursors for synthesizing polymeric materials, and intermediates in the preparation of chemicals for pharmaceutical and scientific industries.

The synthesis of cyclic carbonates from epoxides in accordance with the embodiments described herein reduces carbon dioxide (CO₂) and transforms the gas into high value products such as cyclic carbonates. The epoxide can include R groups at the 1 and 2 positions of the epoxide. The R group can be an alkyl group or an aryl group. In some embodiments, the method of making a cyclic carbonate is described herein, wherein the cyclic carbonate is formed from an epoxide. In some embodiments, the epoxide is propylene oxide. In some embodiments, the epoxide is epichlorohydrin. In some embodiments, the epoxide is styrene oxide. In some embodiments, the epoxide is 1-hexene oxide.

Conventional methods of making cyclic carbonates require highly toxic material such as phosgene which can lead to side products containing a highly corrosive hydrogen chloride. Cyclic carbonates, such as propylene carbonate for example, formed using the catalysts and the methods of the disclosed embodiments, avoids the use of toxic phosgene and therefore provides an environmentally friendly alternative to conventional methods.

### Catalysis and Catalysts

“Catalysis,” as described herein, refers to increasing the rate of a chemical reaction due to the lowering of activation energy. A “catalyst” as described herein, refers to a reagent or a substance that can increase the rate of a chemical reaction of two or more reactants due to its participation in which the catalyzed reaction will have a lower activation energy, whereas without the catalyst, the reaction will not have as high of a reaction rate under same reaction conditions. In some reactions the catalyst can be inhibited, deactivated, or destroyed during a secondary process of the reaction.

Catalysis can be divided into two types of systems, “homogeneous catalyst systems” and “heterogeneous catalyst systems.” Homogeneous catalysts can function in the same phase as the reactants. However, it can be cumbersome to remove the catalyst from the reactants and the product. “Heterogeneous catalyst” refers to the catalyst as a different phase than the reactions, for example a heterogeneous catalyst can be a solid that acts on reactants that are in a liquid phase or a gaseous phase.

Catalysts and methods of making and using the catalysts are described. The catalysts can be recycled after use. In some embodiments, the catalyst can be recovered and recycled for more than once without substantial loss in activity. For example, the catalyst can be recycled for about 10 times, about 20 times, about 30 times, about 40 times, about 50 times, or a higher number of times. In some embodiments, the catalyst can be recovered and recycled for about ten to about fifteen times without substantial loss in activity. The catalyst can for example be a heterogeneous catalyst.

In some embodiments, the catalyst includes at least one polymer quaternary ammonium salt, at least one metal halide and silica gel. Quaternary ammonium salts as described herein, refers to salts of quaternary ammonium cations with an anion. The at least one polymer quaternary ammonium salt, in some embodiments, is polydimethyl diallyl ammonium bromide. The at least one polymer quaternary ammonium salt, in some embodiments, is polydimethyl diallyl ammonium chloride.

A metal halide as described herein, refers to compounds between metals and halogens, and can be prepared by a direct combination of these elements. In some embodiments, the at least one metal halide is ZnBr₂, ZnCl₂, FeCl₃, AlCl₃, NaCl, CaCl₂, Zn(OAc)₂, LiBr or a combination thereof. In some embodiments, the catalyst includes at least one polymer quaternary ammonium salt, at least one metal halide and silica gel, wherein a mass ratio of the metal halide to a total mass including the polymer quaternary ammonium salt, the metal halide, and the silica gel is about 1:200 to about 1:100. In some embodiments, the molar ratio of the polymer quaternary ammonium salt to the metal halide is about 2:1. In some embodiments, the mass ratio of the polymer quaternary ammonium salt to the silica gel is about 1:20 to about 1:5.

In several embodiments described herein, a heterogeneous catalyst is described, in which a polymer quaternary ammonium salt (for example, polydimethyl diallyl ammonium bromide) and a metal halide (for example, zinc bromide) is loaded onto the surface of silica as a carrier. In some embodiments the silica is a silica gel. In several embodiments, the catalyst as described herein allows the yield of cyclic carbonate to be at least about 95% and the catalyst can be reused many times. In some embodiments, the catalyst can be reused more than once without substantial loss in activity. For example, the catalyst can be reused about 10 times, about 20 times, about 30 times, about 40 times, about 50 times, or a higher number of times. In some embodiments, the catalyst can be reused about 10 to about 15 times without substantial loss in activity.

In some embodiments, a method of making the catalyst is described. The method for preparing the catalyst is simple, with the raw materials readily available and inexpensive. Additionally the catalyst can have a high activity rate in relatively mild conditions. In some embodiments, the method of making a cyclic carbonate, for example propylene carbonate, can use the catalyst as described herein. In particular, although the reaction product, cyclic carbonate for example propylene carbonate, is a strong solvent, the catalyst is almost insoluble in it, which accordingly can be easily recovered. In several embodiments, the product can be simply and easily separated from the catalyst, and multiple recycling of the catalyst can be achieved. In the methods described herein, the catalyst is suitable for a broad range of substrates, and can efficiently catalyze the cycloaddition reaction between various epoxy compounds with an epoxy ring located at the end position, such as epoxy chloropropane, hexene oxide and styrene oxide, and carbon dioxide. In some embodiments, the method of making the catalyst includes incubating a first mixture including at least one polymer quaternary ammonium salt, at least one metal halide and a solvent, adding silica gel to the first mixture to form a second mixture, incubating the second mixture and removing the solvent from the second mixture to obtain the catalyst. In some embodiments, the catalyst is a solid. In some embodiments, the method of making a catalyst further includes grinding the catalyst to obtain a powdered form of the catalyst. In some embodiments, incubating the first mixture includes incubating for at least about 10 hours. In some embodiments, incubating the first mixture includes incubating for a time equal to or less than about 18 hours. In some embodiments, incubating the first mixture includes incubating for a time of about 10 hours, about 11 hours, about 12 hours, about 13 hours, about 14 hours, about 15 hours, about 16 hours, about 17 hours, about 18 hours, or a time period between any of these values. In some embodiments, incubating the first mixture includes incubating for a time equal to 12 hours. In some embodiments, the method further includes mixing an organic solvent with water to form the solvent before incubating the first mixture. In some embodiments, the ratio of volume of organic solvent to total volume of the organic solvent and water is about 1:9 to 3:7. In some embodiments, the organic solvent is ethanol. In some embodiments, the organic solvent is methanol. In some embodiments, the at least one polymer quaternary ammonium salt is present in the first mixture at a concentration of at least about 0.01 g/ml. In some embodiments, the at least one polymer quaternary ammonium salt is present in the first mixture at a concentration of about equal to or less than about 0.05 g/ml. In some embodiments, the at least polymer quaternary ammonium salt is present in the first mixture at a concentration of about 0.01 g/ml, about 0.02 g/ml, about 0.03 g/ml, about 0.04 g/ml, about 0.05 g/ml, or a concentration between any of these values. In some embodiments, the at least one polymer quaternary ammonium salt is present in the first mixture at a concentration of about 0.02 g/ml. In some embodiments, the polymer quaternary ammonium salt is polydimethyl diallyl ammonium chloride, polydimethyl diallyl ammonium bromide, or a combination thereof. In some embodiments, wherein the polymer quaternary ammonium salt is polydimethyl diallyl ammonium bromide, and the method further includes subjecting polydimethyl diallyl ammonium chloride to an ion exchanger to obtain the polydimethyl diallyl ammonium bromide before incubating the first mixture. In some embodiments, the metal halide is present in the first mixture at a concentration of at least about 0.010 g/ml. In some embodiments, the metal halide is present in the first mixture at a concentration of less than or equal to about 0.025 g/ml. In some embodiments, the metal halide is present in the first mixture at a concentration of about 0.010 g/ml, about 0.015 g/ml, about 0.020 g/ml, about 0.025 g/ml, or a concentration in between any of these values. In some embodiments, the metal halide is present in the first mixture at a concentration of about 0.015 g/ml. In some embodiments, the metal halide is ZnBr₂, ZnCl₂, FeCl₃, AlCl₃, NaCl, CaCl₂, Zn(OAc)₂ LiBr, or any combination thereof. In some embodiments, incubating the first mixture includes incubating at a temperature of at least about 70° C. In some embodiments, incubating the first mixture includes incubating at a temperature of less than or equal to about 90° C. In some embodiments, incubating the first mixture includes incubating at a temperature of about 80° C. In some embodiments, incubating the first mixture includes incubating at a temperature of about 70° C., about 75° C., about 80° C., about 85° C., about 90° C., or a temperature between any of these values. In some embodiments, the polymer quaternary ammonium salt and the metal halide are present in the first mixture at a molar ratio of at least about 1:4. In some embodiments, polymer quaternary ammonium salt and the metal halide are present in the first mixture at a molar ratio of less than or equal to about 4:1. In some embodiments, polymer quaternary ammonium salt and the metal halide are present in the first mixture at a molar ratio of about 2:1. In some embodiments, polymer quaternary ammonium salt and the metal halide are present in the first mixture at a molar ratio of less than or equal to about 4:1. In some embodiments, polymer quaternary ammonium salt and the metal halide are present in the first mixture at a molar ratio of about 2:1. In some embodiments, the silica gel is present in the second mixture at a concentration of at least about 0.05 g/ ml. In some embodiments, the silica gel is present in the second mixture at a concentration of less than or equal to about 0.25 g/ ml. In some embodiments, the silica gel is present in the second mixture at a concentration of about 0.1 g/ml. In some embodiments, the silica gel is present in the second mixture of about 0.05 g/ml, about 0.10 g/ml, about 0.15 g/ml, about 0.20 g/ml, about 0.25 g/ml, or a concentration between any of these values. In some embodiments, incubating the second mixture includes incubating for at least about 4 hours. In some embodiments, incubating the second mixture includes incubating for a time equal to or less than about 8 hours. In some embodiments, incubating the second mixture includes incubating for about 6 hours. In some embodiments, the incubating the second mixture includes incubating for about 4 hours, about 5 hours, about 6 hours, about 7 hours, about 8 hours, or a time period in between any of these values. In some embodiments, incubating the second reaction mixture includes incubating at a temperature of at least about 70° C. In some embodiments, incubating the second mixture includes incubating at a temperature of less than or equal to about 85° C. In some embodiments, incubating the second mixture includes incubating at a temperature of about 70° C. In some embodiments, incubating the second mixture includes incubating at a temperature of less than or equal to about 85° C. In some embodiments, incubating the second mixture includes incubating at a temperature of about 70° C., about 75° C., about 80° C., about 85° C., or a temperature in between any of these values. In some embodiments, removing the solvent from the second mixture includes aspirating the second mixture. In some embodiments, aspirating the second mixture includes aspirating at a temperature of at least about 70° C. In some embodiments, aspirating the second mixture includes aspirating at a temperature equal to or less than about 120° C. In some embodiments, aspirating the second mixture includes aspirating at a temperature of about 70° C., about 75° C., about 80° C., about 85° C., about 90° C., about 95° C., about 100° C., about 105° C., about 110° C., about 115° C., about 120° C., or a other temperature between any of these values.

### Synthesis of Cyclic Carbonates

Cyclic carbonates as described herein refer to an organic compound that can be produced from an epoxide and carbon dioxide with a catalyst to form a five member ring structure and have attached R groups. Propylene carbonates as described herein refer to an organic compound that can be produced from propylene oxide and carbon dioxide with a catalyst to form a five member ring structure and have attached R groups.

A metal halide as described herein, refers to compounds formed between metals and halogens, and can be prepared by a direct combination of these elements.

Quaternary ammonium salts as described herein, refers to salts of quaternary ammonium cations with an anion.

Methods of making cyclic carbonates, are described herein. In some embodiments, the method includes providing a catalyst, forming a mixture that includes the catalyst and an epoxide, and contacting the mixture with carbon dioxide in a reactor under conditions to form the cyclic carbonate. The catalyst can be as described above, and can include at least one polymer quaternary ammonium salt, at least one metal halide, and silica gel. In some embodiments, the method includes providing a catalyst, forming a mixture that includes the catalyst, propylene oxide, and contacting the mixture with carbon dioxide in a reactor under conditions to form the propylene carbonate.

In some embodiments, the epoxide is propylene oxide, epichlorohydrin, styrene oxide, 1-hexene oxide, or a combination thereof.

In some embodiments, the reactor can have a non-stick lining on at least an inner surface of the reactor. The non-stick lining can reduce or prevent the reactants and products from adhering and localizing at parts of the inner surface of the reactor, which can affect the yield of the reaction. In some embodiments, the non-stick lining includes polytetrafluoroethylene. In some embodiments, the non-stick lining includes a glass liner. In some embodiments, the reactor is a pressure reactor. In some embodiments, the pressure reactor is configured to maintain a pressure of at least about 0 MPa. In some embodiments, the pressure reactor is configured to maintain a pressure equal to or less than about 5MPa. In some embodiments, the pressure reactor is configured to maintain a pressure of about 0 MPa, about 1 MPa, about 2 MPa, about 3 MPa, about 4 MPa, about 5 MPa, or a pressure between any of these values.

In some embodiments, the mixture further includes a solvent. In some embodiments, the solvent is dodecane.

The catalyst may be present in the mixture at an amount dependent on the amount of epoxide used. For example, if more epoxide is used, a larger amount of catalyst will be needed to catalyze the reaction between the epoxide and the carbon dioxide. In some embodiments, the catalyst is present in the mixture at an amount of at least about 5% by weight. In some embodiments, the catalyst is present in the mixture at an amount less than or equal to about 20% by weight. For example, the catalyst is present in the mixture at an amount of about 5%, about 6%, about 7%, about 8%, about 9%, about 10%, about 11%, about 12%, about 13% , about 14%, about 15%, about 16%, about 17%, about 18%, about 19%, about 20% by weight, or a percentage between any of these values. In some embodiments, the epoxide is present in the mixture at an amount of at least about 85% by weight. In some embodiments, the epoxide is present in the mixture at an amount equal to or less than about 95% by weight. In some embodiments, the epoxide is present in the mixture at an amount of about 85%, about 87%, about 89%, about 91%, about 93%, about 95% by weight or a weight percent between any of these values. In some embodiments, the dodecane is present in the mixture at an amount of at least about 5% by weight. In some embodiments, the dodecane is present in the mixture at an amount of about 10% by weight. In some embodiments, the dodecane is present in the mixture at an amount of about 5%, about 6%, about 7%, about 8%, about 9%, about 10% by weight, or aweight percent between any of these values.

In some embodiments, the method further includes adding carbon dioxide into the reactor to displace air in the reactor before the contacting step. In some embodiments, the adding of the carbon dioxide to displace air in the reactor is repeated at least three times. In some embodiments, the carbon dioxide in the contacting step is present in an amount sufficient to provide a pressure of at least about 0.6 MPa in the reactor. In some embodiments, the carbon dioxide in the contacting step is present in an amount sufficient to provide a pressure equal to or less than about 5 MPa. For example, the amount of carbon dioxide can be sufficient to provide a reactor pressure of about 0.6 MPa, about 1 MPa, about 1.5 MPa, about 2 MPa, about 2.5 MPa, about 3 MPa, about 3.5 MPa, about 4 MPa, about 4.5 MPa, about 5 MPa, or a pressure between any of these values. In some embodiments, the carbon dioxide in the contacting step is added by a carbon dioxide high pressure pump.

The mixture and carbon dioxide may be heated to temperatures that can trigger a reaction between the epoxide and the carbon dioxide to form the cyclic carbonate. In some embodiments, the contacting step includes heating to a temperature of at least about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., or a temperature between any of these values. In some embodiments, the contacting step includes heating to a temperature equal to or less than about 150° C. In some embodiments, the contacting step includes heating at a rate of about 5 ° C. per minute to about 6.5° C. per minute. In some embodiments, the contacting step includes heating at a rate of about 5° C. per minute, about 5.5° C. per minute, about 6.0° C. per minute, about 6.5° C. per minute, or a heating rate between any of these values.

The mixture may be stirred to provide an even distribution of the reactants and catalysts within the reactor. In some embodiments, the contacting step includes stiffing at a rate of about 200 rpm to about 300 rpm. In some embodiments, the contacting step includes stiffing at a rate of about 200 rpm, about 210 rpm, about 220 rpm, about 230 rpm, about 240 rpm, about 250 rpm, about 250 rpm, about 260 rpm, about 270 rpm, about 280 rpm, about 290 rpm, about 300 rpm, or a stiffing rate between any of these values. In some embodiments, the contacting step includes contacting the mixture and the carbon dioxide for about 3 hours to about 5 hours. In some embodiments, the contacting step includes contacting the mixture and the carbon dioxide for about 3 hours, about 3.5 hours, about 4 hours, about 4.5 hours, about 5 hours, or a time period between any of these values.

In some embodiments, the method further includes removing unreacted carbon dioxide from the reactor after the contacting step. In some embodiments, removing the unreacted carbon dioxide includes cooling the reactor after the contacting step. In some embodiments, the cooling step includes cooling the reactor to a temperature of about 0° C. In some embodiments, the cooling is performed for about 30 minutes to about 60 minutes. In some embodiments, the cooling is performed for about 30 minutes, about 35 minutes, about 40 minutes, about 45 minutes, about 50 minutes, about 55 minutes, about 60 minutes, or a time period between any of these values.

In some embodiments, the method further includes separating the cyclic carbonate from the mixture after the contacting step. In some embodiments, separating the cyclic carbonate from the mixture includes adding an extraction solvent to dissolve the cyclic carbonate and thus separate the cyclic carbonate from the mixture. In some embodiments, the extraction solvent is ethyl acetate. In some embodiments, the method further includes recovering the catalyst from the mixture. In some embodiments, the recovering step includes subjecting the mixture to at least one of ultrasonic cleaning and centrifuging to separate unreacted epoxide from the catalyst. In some embodiments, the method further includes washing the catalyst with a washing solvent. In some embodiments, the catalyst is washed at least three times. In some embodiments, the method further includes drying the catalyst. In some embodiments, the catalyst is dried under vacuum. In some embodiments, the catalyst is dried at a temperature of about 60° C. to about 90° C. In some embodiments, the catalyst is dried at a temperature of about 60° C., about 65° C., about 70° C., about 75° C., about 80° C., about 85° C., about 90° C. or a temperature between any of these values. In some embodiments, the catalyst is dried for about 1.5 hours to about 3 hours. In some embodiments, the catalyst is dried for about 1.5 hours, about 1.75 hours, about 2 hours, about 2.25 hours, about 2.5 hours, about 2.75 hours, about 3 hours, or a time period between any of these values. In some embodiments, the catalyst can be reused for about 10 times to about 15 times. In some embodiments, the catalyst can be reused for about 10 times.

### EXAMPLES

Additional embodiments are disclosed in further detail in the following examples, which are not in any way intended to limit the scope of the claims.

### Example 1

### Preparation and Synthesis of a Catalyst

Polydimethyl diallyl ammonium chloride (molecular weight, Mw=100000-500000) was subjected to ion exchange to obtain polydimethyl diallyl ammonium bromide. 0.1 g of polydimethyl diallyl ammonium bromide (PDDA-Br) and 0.05 g of zinc bromide (ZnBr₂) were then dissolved in a solvent containing ethanol and water (ethanol: water=3:2, volume ratio), and reacted at 80° C. for 12 hours. After completion of the reaction, 0.5 g of silica gel was added to the above mixed system and immersed at 80° C. for 6 hours The silica was immersed in water, ethanol, PDDA-Br and ZnBr₂. After completion of the reaction, the solvent was aspirated to dry at 80° C., and the solid was ground to obtain a supported catalyst (PDDA—Br—ZnBr₂/SiO₂). In a similar way, the addition amount of ZnBr₂ was changed while the loading amount of PDDA-Br on silica gel remained unchanged to obtain a series of supported catalysts with different molar ratios of PDDA-Br to ZnBr₂ For the experiment, the molar ratios ranged from 0.1 to 6.

### Example 2

### Catalytic Reaction Process and Results

In the experimental operation, all the cycloaddition reactions were performed in a 50 mL high pressure reactor connected with a stainless steel pressure gauge and thermocouple. Specific reaction steps are as follows: 0.1 g of the solid catalyst as prepared above was added into the reactor with a polytetrafluoroethylene lining. 0.7 mL of propylene oxide (10 mmol) and 0.05 g of dodecane were added together into the reactor. A small amount of carbon dioxide was supplied into the reactor to displace the air in the reactor (continuously operated three times), and then a specified amount of carbon dioxide was injected by a carbon dioxide high pressure pump (the pressure was 2.5 MPa under the reaction condition). The reactor was heated and adjusted to an appropriate stiffing speed and heating rate. After a period of reaction at a certain temperature, the reactor was cooled in an ice bath to release the unreacted CO₂, and the product and the substrate were extracted with ethyl acetate. The ethyl acetate dissolves the propylene oxide, in which the propylene oxide is the substrate, and the propylene carbonate is the product. The results are shown in Table 1.

TABLE 1
Effects of the reaction conditions and the ratio of PDDA-Br to
ZnBr2 on the activity of the catalyst PDDA-Br-ZnBr₂/SiO₂, where
the catalyst is at 0.1 g, and the propylene oxide is at 0.7 ml.

| | | | Molar ratio | Conversion | Selectivity |
|---|---|---|---|---|---|
| temp- | | Reaction | of PDDA- | rate of | of |
| erature/ | Reaction | pressure/ | Br to | propylene | propylene |
| ° C. | time/hour | MPa | ZnBr2 | oxide/% | carbonate/% |
| 70 | 5 | 2.5 | 2:1 | 80.3 | 96.9 |
| 80 | 5 | 2.5 | 2:1 | 95.6 | 97.7 |
| 90 | 5 | 2.5 | 2:1 | 97.7 | 96.1 |
| 100 | 5 | 2.5 | 2:1 | 98.1 | 97.6 |
| 110 | 5 | 2.5 | 2:1 | 96.8 | 94.2 |
| 100 | 1 | 2.5 | 2:1 | 75.0 | 95.4 |
| 100 | 2 | 2.5 | 2:1 | 89.3 | 97.1 |
| 100 | 3 | 2.5 | 2:1 | 91.6 | 95.3 |
| 100 | 4 | 2.5 | 2:1 | 97.8 | 97.2 |
| 80 | 5 | 1.0 | 2:1 | 95.1 | 98.5 |
| 80 | 5 | 2.5 | 2:1 | 95.6 | 97.7 |
| 80 | 5 | 3.5 | 2:1 | 95.6 | 98.2 |
| 80 | 5 | 4.0 | 2:1 | 89.5 | 98.5 |
| 100 | 5 | 2.5 | 1:1 | 97.5 | 97.2 |
| 100 | 5 | 2.5 | 1:3 | 91.3 | 97.1 |
| 100 | 5 | 2.5 | 3:1 | 97.2 | 98.3 |

As shown above, the increasing temperature, at a constant time, constant pressure and constant molar ratio, led to a higher conversion rate of propylene oxide to propylene carbonate. The reaction also showed a maximum conversion rate when the reaction time was increased from 1 to 4 hours. A decrease in conversion rate was seen at a reaction pressure of 4.0 MPa. When factors such as temperature, reaction time and pressure were kept constant, and the effect of molar ratio of PDDA-Br to ZnBr₂ was explored, it was shown that the ratios of 1:1 and 3:1 of PDDA-Br gave a higher conversion rate of 97.2% to 97.5% over that of a ratio of 1:3 which gave a conversion rate of 91.3%.

### Example 3

### Separation of the Product and Recycling of the Catalyst

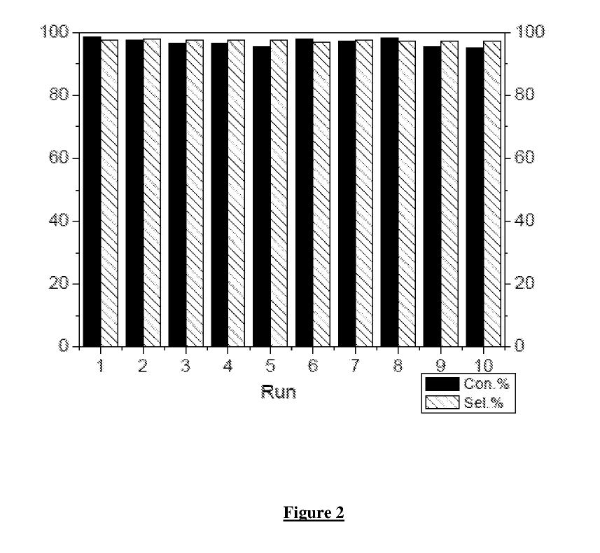

The ethyl acetate after the extraction in Example 2 was poured into the reactor. After simple ultrasonic cleaning and centrifugation, the reacted solution was separated from the solid catalyst, and the upper clear and transparent liquid was used as a sample after the reaction for gas chromatography. The selectivity of the target product was about 97%. The by-product was propane-1,2-diol. The molar ratio of diol and cyclic carbonate was determined by gas chromatograph (GC). The selectivity of cyclic carbonate was denoted as molar percentage of cyclic carbonate in the all products. The ethyl acetate merely acted as solvent to extract product and substrates, and it did not react with anything. The temperature of chromatographic column was kept at 90-120° C., the FID detector at 250° C., and the sample injector at 250° C. Retention time of propylene oxide, propane-1,2-diol (the by-product), and propylene carbonate were 1.14 minutes, 1.40 minutes and 5.60 minutes respectively. The organic product and unreacted substrate adhering to the surface of the catalyst were removed after washing three times with ethyl acetate. The washed catalyst was dried in vacuo at 80 ° C. for 1.5h, and the dried catalyst was directly reused for the next reaction using experimental operations and substrate feeding consistent with the first reaction operation. This catalyst was then reused 10 times, without any decrease in activity and selectivity. The result is shown in FIG. 2. As shown in FIG. 2, the recycling performance of the catalyst is consistent, with the first run to the last, with the conversion rate of propylene oxide above 95% and the selectivity of the propylene carbonate at above 95% as well. For the recycling experiment, the catalyst was provided at 0.1 g, propylene oxide at 0.7 ml, the reaction temperature set at 100° C., and the reaction pressure set at 2.5 MPa, for a reaction time of 5 hours.

With respect to the use of substantially any plural and/or singular terms herein, those having skill in the art can translate from the plural to the singular and/or from the singular to volume of wastewater can be received in the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A catalyst comprising:
at least one polymer quaternary ammonium salt;
at least one metal halide; and
silica gel.

2. The catalyst of claim 1, wherein:
the at least one polymer quaternary ammonium salt is polydimethyl diallyl ammonium bromide, polydimethyl diallyl ammonium chloride or combination thereof; or the at least one metal halide is ZnBr₂, ZnCl₂, FeCl₃, AlCl₃, NaCl, CaCl₂, Zn(OAc)₂, LiBr or a combination thereof.

3. (canceled)

4. (canceled)

5. The catalyst of claim 1, wherein:
a mass ratio of the metal halide to a total mass comprising the polymer quaternary ammonium salt, the metal halide, and the silica gel, is about 1:200 to about 1:100;
a molar ratio of the polymer quarternary ammonium salt to the metal halide is about 2:1; or
a mass ratio of the polymer quarternary ammonium salt to the silica gel is about 1:20 to about 1:5.

6. (canceled)

7. (canceled)

8. A method of making a catalyst, the method comprising:
incubating a first mixture comprising at least one polymer quaternary ammonium salt, at least one metal halide and a solvent;
adding silica gel to the first mixture to form a second mixture;
incubating the second mixture; and
removing the solvent from the second mixture to obtain the catalyst.

9. (canceled)

10. The method of claim 8, further comprising grinding the catalyst to obtain a powdered form of the catalyst.

11. (canceled)

12. (canceled)

13. (canceled)

14. The method of claim 8, further comprising mixing an organic solvent with water to form the solvent before incubating the first mixture.

15. (canceled)

16. (canceled)

17. (canceled)

18. The method of claim 8, wherein the at least one polymer quaternary ammonium salt is present in the first mixture at a concentration of at least about 0.01 g/ml or equal to or less than about 0.05 g/ml.

19. (canceled)

20. (canceled)

21. The method of claim 8, wherein the polymer quaternary ammonium salt is polydimethyl diallyl ammonium chloride, polydimethyl diallyl ammonium bromide, or a combination thereof.

22. (canceled)

23. The method of claim 8, wherein the metal halide is present in the first mixture at a concentration of at least about 0.010 g/ml or less than or equal to about 0.025 g/ml.

24. (canceled)

25. (canceled)

26. The method of claim 8, wherein the metal halide is ZnBr₂, ZnCl₂, FeCl₃, AlCl₃, NaCl, CaCl₂, Zn(OAc)₂ LiBr, or any combination thereof.

27. (canceled)

28. (canceled)

29. (canceled)

30. The method of claim 8, wherein polymer quaternary ammonium salt and the metal halide are present in the first mixture at a molar ratio of at least about 1:4 or less than or equal to about 4:1.

31. (canceled)

32. (canceled)

33. The method of claim 8, wherein the silica gel is present in the second mixture at a concentration of at least about 0.05 g/ml or less than or equal to about 0.25 g/ml.

34. (canceled)

35. (canceled)

36. (canceled)

37. (canceled)

38. (canceled)

39. (canceled)

40. (canceled)

41. (canceled)

42. The method of claim 8, wherein removing the solvent from the second mixture comprises aspirating the second mixture.

43. (canceled)

44. (canceled)

45. A method of making cyclic carbonate, the method comprising:
providing a catalyst, comprising:
at least one polymer quaternary ammonium salt;
at least one metal halide; and
silica gel;
forming a mixture comprising the catalyst and an epoxide; and
contacting the mixture with carbon dioxide in a reactor under conditions to form the cyclic carbonate.

46. (canceled)

47. The method of claim 45, wherein the reactor has a non-stick lining on at least an inner surface of the reactor.

48. (canceled)

49. (canceled)

50. The method of claim 45, wherein reactor is a pressure reactor.

51. (canceled)

52. (canceled)

53. (canceled)

54. (canceled)

55. The method of claim 45, wherein the catalyst is present in the mixture at an amount of at least about 5% by weight or less than or equal to about 20% by weight.

56. (canceled)

57. The method of claim 45, wherein the epoxide is present in the mixture at an amount of at least about 85% by weight or equal to or less than about 95% by weight.

58. (canceled)

59. (canceled)

60. (canceled)

61. The method of claim 45, further comprising adding carbon dioxide into the reactor to displace air in the reactor before the contacting step.

62. (canceled)

63. (canceled)

64. (canceled)

65. The method of claim 45, wherein the carbon dioxide in the contacting step is added by a carbon dioxide high pressure pump.

66. The method of claim 45, wherein the contacting step comprises heating to a temperature of at least about 70° C. or equal to or less than about 150° C.

67. (canceled)

68. (canceled)

69. (canceled)

70. (canceled)

71. The method of claim 45, further comprising removing unreacted carbon dioxide after the contacting step.

72. (canceled)

73. (canceled)

74. (canceled)

75. The method of claim 45, further comprising separating the cyclic carbonate from the mixture after the contacting step.

76. The method of claim 75, wherein separating the cyclic carbonate from the mixture comprises adding an extraction solvent to dissolve the epoxide and thus separate the cyclic carbonate from the mixture.

77. (canceled)

78. The method of claim 76, further comprising recovering the catalyst from the mixture.

79. The method of claim 78, wherein the recovering step comprises subjecting the mixture to at least one of ultrasonic cleaning and centrifuging to separate unreacted epoxide from the catalyst.

80. The method of claim 78, further comprising washing the catalyst with a washing solvent.

81. (canceled)

82. The method of claim 80, further comprising drying the catalyst.

83. (canceled)

84. (canceled)

85. (canceled)

86. (canceled)

87. (canceled)
